SEQUENCE LISTING (35 sequences)

SEQ ID NO: 1 - protein, 36 aa, Coronavirus SARS-CoV-2
DISGINASVVNIQKEIDRLNEVAKNLNESLIDLQEL

SEQ ID NO: 2 - protein, 36 aa, synthetic construct
DISQINASVVNIEYEIKKLEEVAKKLEESLIDLQEL

SEQ ID NO: 3 - protein, 36 aa, synthetic construct
SIDQINATFVDIEYEIKKLEEVAKKLEESYIDLKEL

SEQ ID NO: 4 - protein, 36 aa, Coronavirus HCoV-OC43
SLDQINVTFLDLEYEMKKLEEAIKKLEESYIDLKEL

SEQ ID NO: 5 - protein, 42 aa, synthetic construct
  MOD_RES 1: note = Beta-amino acid analog
  MOD_RES 32: note = Beta-amino acid analog
DISQINASVVNIEYEIKKLEEVAKKLEESLIDLQELGSGSGC

SEQ ID NO: 6 - protein, 42 aa, synthetic construct; identical to SEQ ID NO: 5
  MOD_RES 8: note = Beta-amino acid analog
  MOD_RES 32: note = Beta-amino acid analog

SEQ ID NO: 7 - protein, 42 aa, synthetic construct; identical to SEQ ID NO: 5
  MOD_RES 1: note = Beta-amino acid analog
  MOD_RES 4: note = Beta-amino acid analog
  MOD_RES 32: note = Beta-amino acid analog
  MOD_RES 34: note = Beta-amino acid analog

SEQ ID NO: 8 - protein, 42 aa, synthetic construct; identical to SEQ ID NO: 5
  MOD_RES 1: note = Beta-amino acid analog
  MOD_RES 4: note = Beta-amino acid analog
  MOD_RES 32: note = Beta-amino acid analog
  MOD_RES 35: note = Beta-amino acid analog

SEQ ID NO: 9 - protein, 42 aa, synthetic construct; identical to SEQ ID NO: 5
  MOD_RES 1: note = Beta-amino acid analog
  MOD_RES 8: note = Beta-amino acid analog
  MOD_RES 32: note = Beta-amino acid analog
  MOD_RES 35: note = Beta-amino acid analog

SEQ ID NO: 10 - protein, 42 aa, synthetic construct
  MOD_RES 17: note = 3-aminopyrrolidine-4-carboxylic acid
  MOD_RES 21: note = Beta-amino acid analog
  MOD_RES 24: note = 3-aminopyrrolidine-4-carboxylic acid
  MOD_RES 28: note = Beta-amino acid analog
DISQINASVVNIEYEIZKLEEVAZKLEESLIDLQELGSGSGC

SEQ ID NO: 11 - protein, 42 aa, synthetic construct
  MOD_RES 18: note = 3-aminopyrrolidine-4-carboxylic acid
  MOD_RES 21: note = Beta-amino acid analog
  MOD_RES 25: note = 3-aminopyrrolidine-4-carboxylic acid
  MOD_RES 28: note = Beta-amino acid analog
DISQINASVVNIEYEIKZLEEVAKZLEESLIDLQELGSGSGC

SEQ ID NO: 12 - protein, 42 aa, synthetic construct; identical to SEQ ID NO: 5
  MOD_RES 18: note = Beta-amino acid analog
  MOD_RES 21: note = Beta-amino acid analog
  MOD_RES 25: note = Beta-amino acid analog
  MOD_RES 28: note = Beta-amino acid analog

SEQ ID NO: 13 - protein, 42 aa, synthetic construct
  MOD_RES 18: note = 3-aminopyrrolidine-4-carboxylic acid
  MOD_RES 21: note = U
  MOD_RES 25: note = 3-aminopyrrolidine-4-carboxylic acid
  MOD_RES 28: note = U
DISQINASVVNIEYEIKZLEUVAKZLEUSLIDLQELGSGSGC

SEQ ID NO: 14 - protein, 42 aa, synthetic construct
  MOD_RES 21: note = Beta-amino acid analog
  MOD_RES 24: note = 3-aminopyrrolidine-4-carboxylic acid
  MOD_RES 28: note = Beta-amino acid analog
DISQINASVVNIEYEIKKLEEVAZKLEESLIDLQELGSGSGC

SEQ ID NO: 15 - protein, 42 aa, synthetic construct; identical to SEQ ID NO: 10
  MOD_RES 17: note = 3-aminopyrrolidine-4-carboxylic acid
  MOD_RES 24: note = 3-aminopyrrolidine-4-carboxylic acid
  MOD_RES 28: note = Beta-amino acid analog

SEQ ID NO: 16 - protein, 42 aa, synthetic construct
  MOD_RES 17: note = 3-aminopyrrolidine-4-carboxylic acid
  MOD_RES 21: note = Beta-amino acid analog
  MOD_RES 28: note = Beta-amino acid analog
DISQINASVVNIEYEIZKLEEVAKKLEESLIDLQELGSGSGC

SEQ ID NO: 17 - protein, 42 aa, synthetic construct; identical to SEQ ID NO: 10
  MOD_RES 17: note = 3-aminopyrrolidine-4-carboxylic acid
  MOD_RES 21: note = Beta-amino acid analog
  MOD_RES 24: note = 3-aminopyrrolidine-4-carboxylic acid

SEQ ID NO: 18 - protein, 42 aa, synthetic construct; identical to SEQ ID NO: 10
  MOD_RES 1: note = Beta-amino acid analog
  MOD_RES 4: note = Beta-amino acid analog
  MOD_RES 17: note = 3-aminopyrrolidine-4-carboxylic acid
  MOD_RES 21: note = Beta-amino acid analog
  MOD_RES 24: note = 3-aminopyrrolidine-4-carboxylic acid
  MOD_RES 28: note = Beta-amino acid analog
  MOD_RES 32: note = Beta-amino acid analog
  MOD_RES 34: note = Beta-amino acid analog

SEQ ID NO: 19 - protein, 42 aa, synthetic construct
  MOD_RES 1: note = Beta-amino acid analog
  MOD_RES 4: note = Beta-amino acid analog
  MOD_RES 17: note = 3-aminopyrrolidine-4-carboxylic acid
  MOD_RES 21: note = Beta-amino acid analog
  MOD_RES 25: note = 3-aminopyrrolidine-4-carboxylic acid
  MOD_RES 28: note = Beta-amino acid analog
  MOD_RES 32: note = Beta-amino acid analog
  MOD_RES 34: note = Beta-amino acid analog
DISQINASVVNIEYEIZKLEEVAKZLEESLIDLQELGSGSGC

SEQ ID NO: 20 - protein, 42 aa, synthetic construct; identical to SEQ ID NO: 10
  MOD_RES 1: note = Beta-amino acid analog
  MOD_RES 4: note = Beta-amino acid analog
  MOD_RES 9: note = Beta-amino acid analog
  MOD_RES 17: note = 3-aminopyrrolidine-4-carboxylic acid
  MOD_RES 21: note = Beta-amino acid analog
  MOD_RES 24: note = 3-aminopyrrolidine-4-carboxylic acid
  MOD_RES 28: note = Beta-amino acid analog
  MOD_RES 32: note = Beta-amino acid analog
  MOD_RES 34: note = Beta-amino acid analog

SEQ ID NO: 21 - protein, 42 aa, synthetic construct; identical to SEQ ID NO: 10
  MOD_RES 1: note = Beta-amino acid analog
  MOD_RES 4: note = Beta-amino acid analog
  MOD_RES 9: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 28: note = Beta-amino acid analog
  MOD_RES 32: note = Beta-amino acid analog
  MOD_RES 34: note = Beta-amino acid analog

SEQ ID NO: 22 - protein, 42 aa, synthetic construct; identical to SEQ ID NO: 16
  MOD_RES 1: note = Beta-amino acid analog
  MOD_RES 4: note = Beta-amino acid analog
  MOD_RES 17: note = 3-aminopyrrolidine-4-carboxylic acid
  MOD_RES 32: note = Beta-amino acid analog
  MOD_RES 34: note = Beta-amino acid analog

SEQ ID NO: 23 - protein, 42 aa, synthetic construct; identical to SEQ ID NO: 14
  MOD_RES 1: note = Beta-amino acid analog
  MOD_RES 4: note = Beta-amino acid analog
  MOD_RES 24: note = 3-aminopyrrolidine-4-carboxylic acid
  MOD_RES 32: note = Beta-amino acid analog
  MOD_RES 34: note = Beta-amino acid analog

SEQ ID NO: 24 - protein, 42 aa, synthetic construct; identical to SEQ ID NO: 10
  MOD_RES 1: note = Beta-amino acid analog
  MOD_RES 4: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog

SEQ ID NO: 25 - protein, 42 aa, synthetic construct; identical to SEQ ID NO: 10
  MOD_RES 1: note = Beta-amino acid analog
  MOD_RES 4: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 28: note = Beta-amino acid analog
  MOD_RES 32: note = Beta-amino acid analog
  MOD_RES 34: note = Beta-amino acid analog

SEQ ID NO: 26 - protein, 42 aa, synthetic construct; identical to SEQ ID NO: 10
  MOD_RES 1: note = Beta-amino acid analog
  MOD_RES 4: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 39: note = Beta-amino acid analog
  MOD_RES 41: note = Beta-amino acid analog

SEQ ID NO: 27 - protein, 42 aa, synthetic construct; identical to SEQ ID NO: 10
  MOD_RES 1: note = Beta-amino acid analog
  MOD_RES 4: note = Beta-amino acid analog
  MOD_RES 7: note = 2-aminoisobutyric acid
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 39: note = Beta-amino acid analog
  MOD_RES 41: note = Beta-amino acid analog

SEQ ID NO: 28 - protein, 42 aa, synthetic construct
  MOD_RES 1: note = Beta-amino acid analog
  MOD_RES 4: note = Beta-amino acid analog
  MOD_RES 9: note = 2-aminoisobutyric acid
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 39: note = Beta-amino acid analog
  MOD_RES 41: note = Beta-amino acid analog
DISQINASAVNIEYEIZKLEEVAZKLEESLIDLQELGSGSGC

SEQ ID NO: 29 - protein, 42 aa, synthetic construct; identical to SEQ ID NO: 10
  MOD_RES 1: note = Beta-amino acid analog
  MOD_RES 4: note = Beta-amino acid analog
  MOD_RES 10: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 37: note = Beta-amino acid analog
  MOD_RES 41: note = Beta-amino acid analog

SEQ ID NO: 30 - protein, 42 aa, synthetic construct; identical to SEQ ID NO: 10
  MOD_RES 1: note = Beta-amino acid analog
  MOD_RES 4: note = Beta-amino acid analog
  MOD_RES 8: note = Beta-amino acid analog
  MOD_RES 11: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 37: note = Beta-amino acid analog
  MOD_RES 41: note = Beta-amino acid analog

SEQ ID NO: 31 - protein, 42 aa, synthetic construct; identical to SEQ ID NO: 10
  MOD_RES 1: note = Beta-amino acid analog
  MOD_RES 4: note = Beta-amino acid analog
  MOD_RES 12: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 37: note = Beta-amino acid analog
  MOD_RES 41: note = Beta-amino acid analog

SEQ ID NO: 32 - protein, 42 aa, synthetic construct; identical to SEQ ID NO: 10
  MOD_RES 1: note = Beta-amino acid analog
  MOD_RES 4: note = Beta-amino acid analog
  MOD_RES 13: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 37: note = Beta-amino acid analog
  MOD_RES 41: note = Beta-amino acid analog

SEQ ID NO: 33 - protein, 42 aa, synthetic construct
  MOD_RES 1: note = Beta-amino acid analog
  MOD_RES 4: note = Beta-amino acid analog
  MOD_RES 8: note = Beta-amino acid analog
  MOD_RES 13: note = 2-aminoisobutyric acid
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 37: note = Beta-amino acid analog
  MOD_RES 41: note = Beta-amino acid analog
DISQINASVVNIAYEIZKLEEVAZKLEESLIDLQELGSGSGC

SEQ ID NO: 34 - protein, 42 aa, synthetic construct; identical to SEQ ID NO: 10
  MOD_RES 1: note = Beta-amino acid analog
  MOD_RES 4: note = Beta-amino acid analog
  MOD_RES 17: note = 3-aminopyrrolidine-4-carboxylic acid
  MOD_RES 24: note = 3-aminopyrrolidine-4-carboxylic acid
  MOD_RES 32: note = Beta-amino acid analog
  MOD_RES 34: note = Beta-amino acid analog
  MOD_RES 37: note = Beta-amino acid analog
  MOD_RES 41: note = Beta-amino acid analog

SEQ ID NO: 35 - protein, 6 aa, synthetic construct
GSGSGC

(19) United States
(12) Patent Application Publication
(10) Pub. No.: US 2026/0232759 A1
(43) Pub. Date: Aug. 13, 2026

(54) PEPTIDES THAT INHIBIT INFECTION BY SARS-COV-2, THE VIRUS THAT CAUSES COVID-19 DISEASE

(71) Applicants: Wisconsin Alumni Research Foundation, Madison, WI (US); THE TRUSTEES OF COLUMBIA UNIVERSITY IN THE CITY OF NEW YORK NEW YORK, New York, NY (US)

(72) Inventors: Samuel Gellman, Madison, WI (US); Victor Outlaw, Madison, WI (US); Anne Moscona, New York, NY (US); Matteo Porotto, New York, NY (US); Zhen Yu, Madison, WI (US)

(73) Assignees: Wisconsin Alumni Research Foundation, Madison, WI (US); THE TRUSTEES OF COLUMBIA UNIVERSITY IN THE CITY OF NEW YORK, New York, NY (US)

(21) Appl. No.: 18/703,242

(22) PCT Filed: Oct. 21, 2022
(86) PCT No.: PCT/US2022/047449

§ 371 Date: Apr. 19, 2024

Related U.S. Application Data
(60) Provisional application No. 63/270,851, filed on Oct. 22, 2021.

(51) Int. Cl. A61K 38/16; A61P 31/14

(52) U.S. Cl. CPC A61K 38/162; A61P 31/14

(57) ABSTRACT

Anti-SARS-CoV-2 α/β-polypeptides, pharmaceutical compositions containing the same, and methods to inhibit, treat, and ameliorate SARS-CoV-2 infections in mammals, including humans.

### CROSS-REFERENCE TO RELATED APPLICATIONS

This is a national phase of PCT Application Ser. No. PCT/US2022/047449, filed Oct. 21, 2022, which claims priority to provisional application Ser. No. 63/270,851, filed Oct. 22, 2021, which is incorporated herein by reference.

### FEDERAL FUNDING STATEMENT

This invention was made with government support under All 14736, GM056414, and AI121349 awarded by the National Institutes of Health. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Sep. 18, 2024, is named “REPLACEMENT_SEQ_LIST—09824502-P210396US02.xml” and is 101,736 bytes in size.

### BACKGROUND

SARS-CoV-2 (“CoV2”) is an “enveloped” virus: each viral particle is surrounded by a membrane known as an “envelope.” Infection cannot occur until the viral envelope is fused with the host cell membrane. Fusion allows transfer of the viral genome into the cell's cytoplasm. CoV2 uses a type-I mechanism to induce membrane fusion. Fusion is driven by a single trimeric protein (Spike, or “S”) on the surface of the viral particle. The fusion process by which the virus gains entry to the cell is shown schematically in FIG. 1. In the fusion process, the S protein trimer undergoes profound conformational changes that drive membrane fusion (FIG. 2). A transient form of the S protein is rearranged to a more stable and compact “six-helix bundle” (6HB). The 6HB formation provides the driving force for fusion of the host cell membrane and the viral envelop.

This mechanism of viral fusion is not unique to CoV2. Many enveloped pathogenic viruses employ a type-I mechanism for cellular infection. While the proteins that orchestrate the membrane fusion process differ among these viruses, the basic principles of the fusion mechanism are analogous among these viruses. FIG. 3 show that 6HB is formed in HIV and HIPV3 fusion proteins similarly as CoV2. In HIV, for example, the key protein that drives viral fusion is gp41. Enfuvirtide, a drug to treat AIDS, has as its active agent a 36-residue peptide derived the CHR domain of gp41. Enfuvirtide inhibits HIV infection by blocking the formation of the 6HB that is needed to fuse the virus to the cell. This inhibitory mechanism is shown schematically in FIG. 4.

Enfuvirtide must be administered by injection, twice a day, for life. This onerous dosing schedule has limited clinical use. But this frequent dosing, by injection, is required because enfuvirtide is very rapidly degraded in the bloodstream. Rapid destruction by proteases is a common liability of conventional peptide drugs. Peptides that contain only proteinogenic, alpha-amino acid residues are natural substrates for proteases and thus typically have very short half-lives.

The in vivo stability of polypeptide drugs can be improved by substituting non-natural amino acid residues into the sequence of the polypeptide. See, for example, “Structural and Biological Mimicry of Protein Surface Recognition by a/b-Peptide Foldamers,” W. S. Home, L. M. Johnson, T. J. Ketas, P. J. Klasse, M. Lu, J. P. Moore and S. H. Gellman Proc. Natl. Acad. Sci. USA 2009, 106, 14751 and “Enhancement of a-Helix Mimicry by an a/b-Peptide Foldamer via Incorporation of a Dense Ionic Side Chain Array,” L. M. Johnson, D. E. Mortenson, H. G. Yun, W. S. Home, T. J. Ketas, M. Lu, J. P. Moore and S. H. Gellman J. Am. Chem. Soc. 2012, 134, 7317. See also U.S. Pat. No. 10,723,779 to Gellman et al., U.S. Pat. No. 10,647,743 to Home et al., and 10,501,518, to Gellman et al.

### SUMMARY

Disclosed herein are polypeptide compounds that inhibit the infectivity of CoV2. The peptide compounds include non-natural β-amino acid residues (which may or may not be cyclically constrained). The presence of these β-amino acid residues renders the compounds resistant to proteolysis in vivo, thus improving their pharmacological activity. The peptide backbone of the subject compounds has been altered by replacing an α-amino acid residue with a β-amino acid residue. Backbone modification to include β-amino acid residues profoundly diminishes susceptibility to cleavage by proteases.

The structure of the Spike protein 6HB bundle in CoV2 is known. Disclosed herein is a peptide modeled on the HR2 domain of the CoV2 Spike protein which is potent inhibitor of cellular fusion mediated by the Spike protein.

Disclosed herein are modified versions of the SARS-CoV-2 HR2 peptide that have improved solubility. These compounds, which bear a cholesterol moiety, display potent inhibition of Spike protein-mediated cellular fusion. The subject compounds inhibit CoV2 infection in humans, and are also effective to treat CoV2-infected humans, and are longer-lasting in vivo due to their resistance to degradation by proteolytic enzymes.

Thus, disclosed herein are the following: A composition of matter comprising a polypeptide as shown in SEQ ID NO: 2, or a polypeptide with at least 80%, 85%, 90%, or 95%, but less than 100% sequence identity to SEQ ID NO: 2, wherein at least one α-amino acid residue in the polypeptide is replaced with a β-amino acid residue.

In certain aspects, from 1 to 10 α-amino acid residues in the polypeptide are replaced with a β-amino acid residue.

In certain aspects, at least one α-amino acid residue in the polypeptide is replaced with a cyclically constrained β-amino acid residue.

In some embodiments, at least one α-amino acid residue in the polypeptide is replaced with a cyclically constrained β-amino acid residue selected from the group consisting of 2-aminocyclopentane carboxylic acid and β-aminopyrrolidine-4-carboxylic acid.

In some embodiments, at least one α-amino acid residue in the polypeptide is replaced with a 2-aminoisobutyric acid.

In certain aspects, the polypeptide further comprises a lipid moiety.

In certain aspects, the polypeptide further comprises at least one poly(ethylene glycol) moiety.

In certain aspects, the polypeptide further comprises a lipid moiety and at least one poly(ethylene glycol) moiety.

The lipid moiety is attached to a terminus of the polypeptide.

In some embodiments, the lipid moiety is selected from the group consisting of cholesterol, tocopherol, and palmitate.

In certain aspects, the polypeptide comprises a compound selected from the group consisting of SEQ ID NOs: 5-34.

Also disclosed herein is a composition of matter comprising SEQ ID NO: 34, or a polypeptide with at least 80%, 85%, 90%, or 95%, but less than 100% sequence identity to SEQ ID NOs: 7, 23, 24, 32, and 34.

Also disclosed herein is a method to inhibit infection by CoV2 in a mammalian subject, including a human subject, the method comprising administering to the subject a CoV2 infection-inhibiting amount of a composition of matter according to the present disclosure.

Also disclosed herein is a method to ameliorate symptoms of CoV2 infection in a mammalian subject, including a human subject, the method comprising administering to the subject a CoV2 symptom-ameliorating amount of a composition of matter according to the present disclosure.

Also disclosed herein is a pharmaceutical composition comprising a composition of matter according to the present disclosure, in combination with a pharmaceutically suitable carrier.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 is a schematic diagram of an enveloped virus infecting a cell using a viral fusion protein (itself a trimer) to create an entry pore into the cell.

FIG. 2 depicts a pre-fusion model of the SARS-CoV-2 spike protein (on the left side of FIG. 2) and a post-fusion model of the same protein (on the right side of FIG. 2), noting that the protein must go through multiple conformational changes to yield viral infection.

FIG. 3 depicts models of HIV, HIPV3, and SARS-CoV-2 fusion proteins, showing that six-helix bundles appear to be common among pathogenic viruses.

FIG. 4 is a schematic diagram depicting interruption of the pore-formation process using an agent that inhibits the viral fusion protein trimers from forming the six-helix bundles that are needed to complete the process. The AIDS drug Enfuvirtide is thought to act via this mechanism.

FIG. 5 shows the results of inhibition of CoV2 spread (ex vivo) by the native CoV2 HRC peptide (SEQ ID NO: 1) using a human airway epithelium (“HAE”) test method (See the Example section for the method). The peptide has an added C-terminal cholesterol moiety. Spread of fluorescent virus (light dots) is shown at the indicated days with or without peptide treatment.

FIG. 6 depicts inhibition of cell fusion and cell toxicity of Peptide 1 (SEQ ID NO: 2; -▪-) modified from the native CoV2 HRC peptide (SEQ ID NO: 1; [symbol] ) and also compared to EK1 (SEQ ID NO: 4; -•-). The EK1 sequence is derived from the human coronavirus HCoV-OC43 HRC domain and has been reported to display inhibition to CoV2 infection. Peptide 1 and EK1 have improved solubility compared to the native CoV2 HRC peptide. The native CoV2 HRC peptide, Peptide 1 and EK1 contain only α-amino acids.

FIG. 7 depicts possible residue mutations that might be incorporated into a viral fusion protein inhibitor to increase its potency and half-life.

FIG. 8 shows an exemplary anti-CoV2 polypeptide according to the present disclosure. The polypeptide include aliphatic β-amino acid substitutions and cyclically constrained β-amino acid substitutions, along with a C-terminal poly(ethylene glycol)-cholesterol “tail.”

FIG. 9 shows inhibition of cell-cell S-protein-mediated fusion by exemplary anti-CoV2 polypeptides according to the present disclosure. The polypeptides all have a C-terminal poly(ethylene glycol)-cholesterol “tail” added through a “GSGSGC” linker. “VKO5144-SARS2 HRC QE 5 peg4 chol”: SEQ ID NO: 22; “VKO5146-SARS2 HRC QE 6 peg4 chol”: SEQ ID NO: 23; “VKO5148-SARS2 HRC QE 7 peg4 chol”: SEQ ID NO: 24; “VK05150-SARS2 HRC QE 8 peg4 chol”: SEQ ID NO: 25; “SARS mod peg 4 chol dimer”: Control.

FIGS. 10A-10C shows IC₅₀ of exemplary anti-CoV2 polypeptides to inhibit cell-cell S-protein-mediated fusion. Amino acid residues highlighted by an oval other than “Z” represent β²- or β³-amino acid residues that share the same sidechain as their α-amino acid analogs. “Z” highlighted by an oval is β-aminopyrrolidine-4-carboxylic acid (also known as “APC”), which may or may not be protonated.

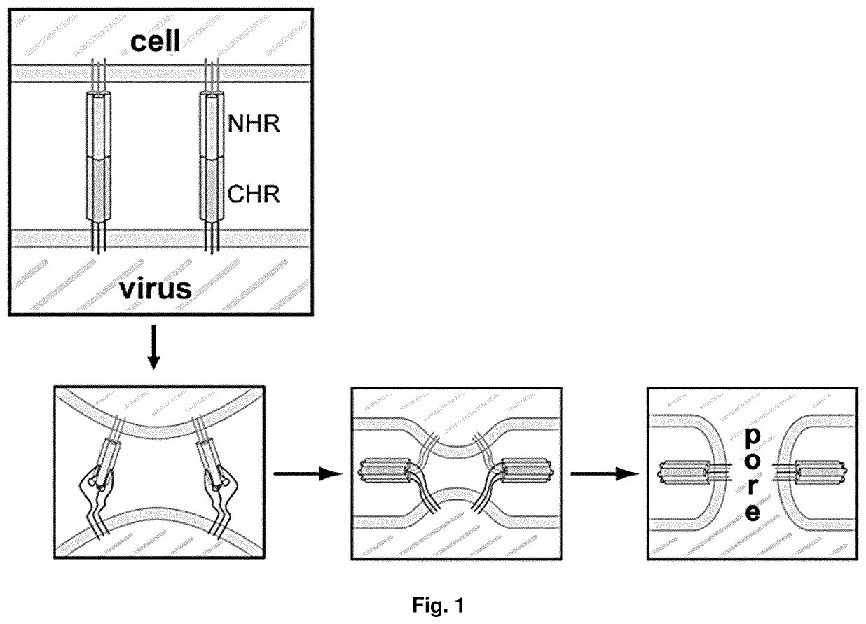

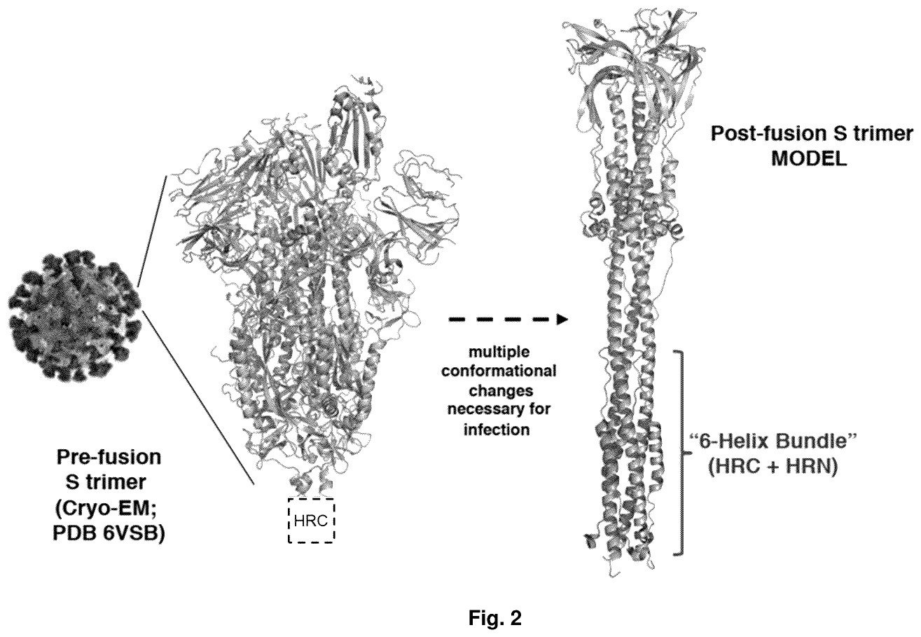

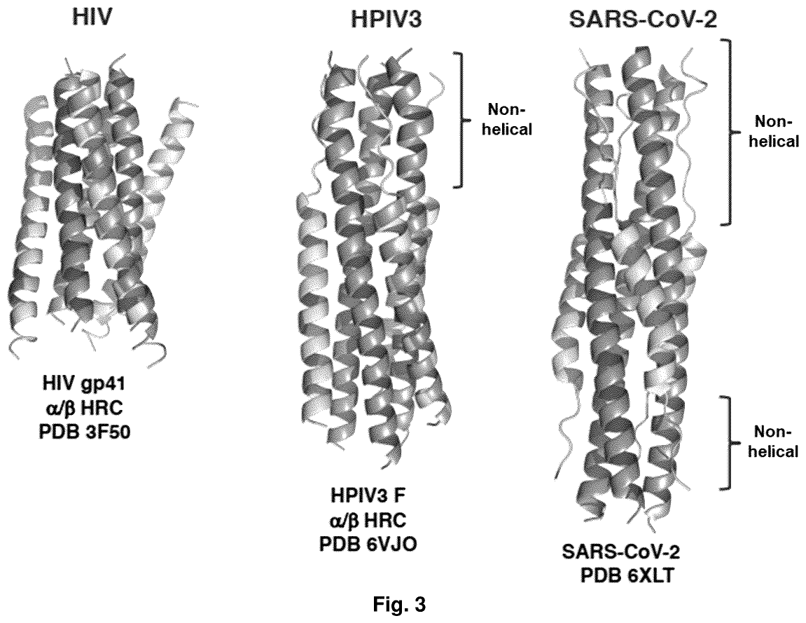

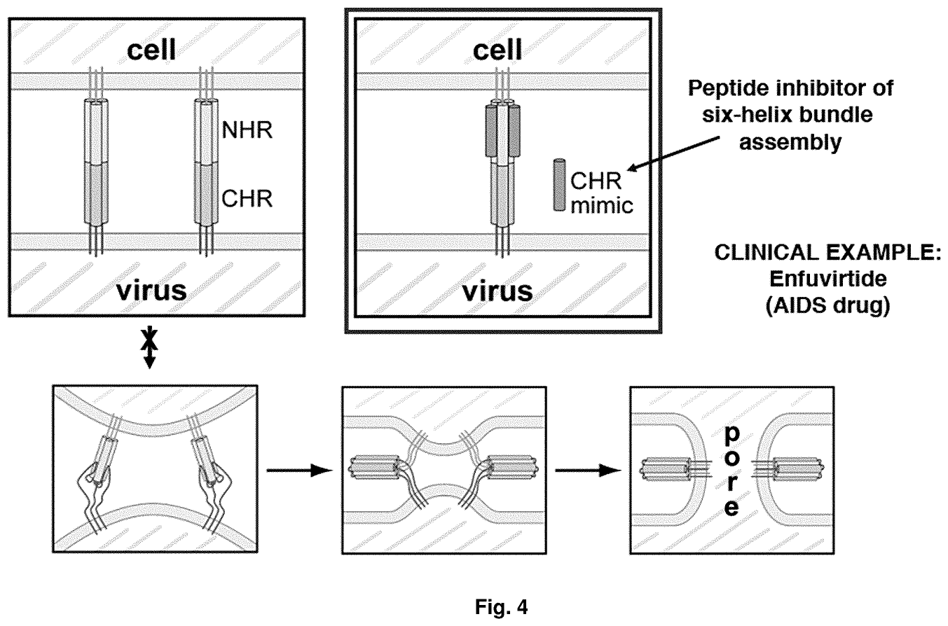

### DETAILED DESCRIPTION

### Abbreviations and Definitions

ACPC=2-aminocyclopentane carboxylic acid.
Aib=2-aminoisobutyric acid (i.e., 2-methylalanine)
APC=β-aminopyrrolidine-4-carboxylic acid.

“Cyclically constrained” when referring to a β-amino acid or β-amino acid residue means a β-amino acid or β-amino acid residue in which the α-position and β-position carbon atoms in the backbone of the β-amino acid are incorporated into a substituted or unsubstituted C₄ to C₁₀ cycloalkyl, cycloalkenyl, or heterocycle moiety, wherein heterocycle moieties may have 1, 2, or 3 heteroatoms selected from the group consisting of N, S, and O. Generally preferred cyclically constrained -amino acids have the α-position and -position carbon atoms in the backbone incorporated into a substituted or unsubstituted C₅ to C₈ cycloalkyl, cycloalkenyl, or heterocycle moiety having one or more N, S, or O atoms as the heteroatom. Within any given anti-CoV2 peptide disclosed herein, the cyclically constrained β-amino acid residues may be the same or different.

The amino acid residues in the compounds disclosed herein may either be present in their D or their L configuration. The terms “peptide” and “polypeptide” are used synonymously and refer to a polymer of amino acids which are linked via an amide linkage.

The terms “identical” or percent “identity” refer to two of more sequences that are the same or have a specified percentage of amino acid residues that are the same, when compared and aligned for maximum correspondence, as measured using one of the following sequence comparison algorithms or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Nat. Acad. Sci., USA, 85: 2444, by computerized implementations of these algorithms or by visual inspection.

“Pharmaceutically suitable salts” means salts formed with acids or bases the addition of which does not have undesirable effects when administered to mammals, including humans. Preferred are the salts with acids or bases listed in the U.S. Pharmacopoeia (or any other generally recognized pharmacopoeia) for use in humans. A host of pharmaceutically suitable salts are well known in the art. For basic active ingredients, all acid addition salts are useful as sources of the free base form even if the particular salt, per se, is desired only as an intermediate product as, for example, when the salt is formed only for purposes of purification, and identification, or when it is used as intermediate in preparing a pharmaceutically suitable salt by ion exchange procedures. Pharmaceutically-suitable salts include, without limitation, those derived from mineral acids and organic acids, explicitly including hydrohalides, e.g., hydrochlorides and hydrobromides, sulphates, phosphates, nitrates, sulphamates, acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methane sulphonates, ethanesulphonates, benzenesulphonates, p-toluenesulphonates, cyclohexylsulphamates, quinates, and the like. Base addition salts include those derived from alkali or alkaline earth metal bases or conventional organic bases, such as triethylamine, pyridine, piperidine, morpholine, N methylmorpholine, and the like. Other suitable salts are found in, for example, “Handbook of Pharmaceutical Salts: Properties, Selection, and Use, 2nd Ed.” P. H. Stahl and C. G. Wermuch, Eds., © 2011, Wiley-VCH (ISBN-13: 978-3906390512) and “Pharmaceutical Salts and Co-Crystals,” Johan Wouters, Editor, © 2011, The Royal Society of Chemistry (U.K.) (ISBN-13: 978-1849731584).

“Treating” or “treatment” of a condition as used herein may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods of the present invention can comprise, consist of, or consist essentially of the essential elements and limitations of the method described herein, as well as any additional or optional ingredients, components, or limitations described herein or otherwise useful in synthetic organic chemistry, pharmacy, pharmacology, and the like.

Compounds that Inhibit CoV2 Infection

Disclosed herein is a composition of matter comprising polypeptide compounds that inhibit the infectivity of CoV2. The peptides mimic a portion of the Spike protein of CoV2 and bind to a transient form of the trimer that occurs during the infection process. Peptide binding prevents rearrangement of the transient form of S to a more stable and compact “six-helix bundle” (6HB); in the absence of the peptide, 6HB formation provides the driving force for fusion of the host cell membrane and the viral envelope.

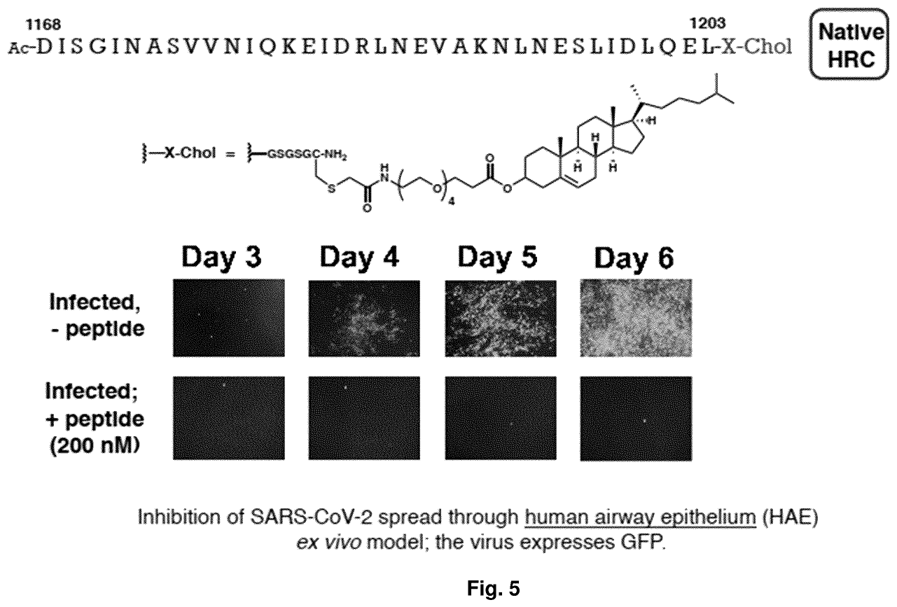

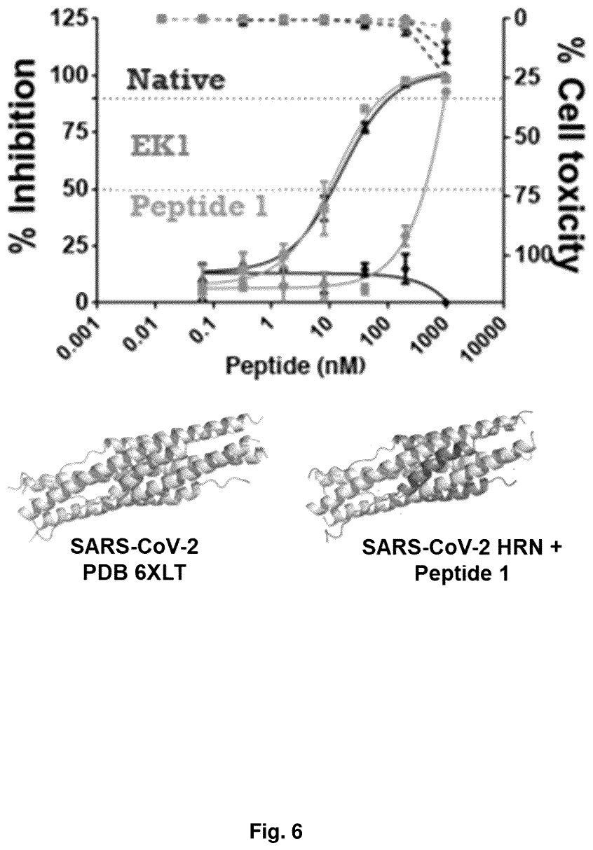

The structure of the Spike protein 6HB bundle in CoV2 is known, which provides molecular target for designing the peptides. The peptides are modeled on the HR2 domain of the CoV2 Spike protein. It has been found that a 36-residue peptide corresponding to residues 1168 to 1203 within the HRC domain of the CoV2 S protein (SEQ ID NO: 1) is a potent inhibitor of cellular fusion mediated by the S protein. See FIG. 5 and Outlaw et al., mBio 2020, 11: e01935-20. However, this peptide is extremely challenging to produce because of low solubility. Modified versions of the CoV2 HR2 peptide were designed to display improved solubility (SEQ ID NOs: 2-4). See FIG. 6, WO2021/216891 A2 and Outlaw et al., mBio 2020, 11: e01935-20. The modified peptides, comprising entirely of α-amino acids, display potent inhibition of S-mediated cellular fusion with activities comparable to the activity of the native peptide, but rapidly degraded by proteases.

Thus, disclosed herein are polypeptide compounds that include non-natural β-amino acid residues. The presence of these β-amino acid residues renders the compounds resistant to proteolysis in vivo, thus improving their pharmacological activity.

In preferred versions, the polypeptide has an amino acid sequence of SEQ ID NO: 2, or at least 80%, 85%, 90%, or 95%, but less than 100% sequence identity to SEQ ID NO: 2, wherein at least one α-amino acid residue in the polypeptide is replaced with a β-amino acid residue.

In various embodiments, the polypeptide has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 96%, or at least 97% sequence identity to SEQ ID NO: 2, wherein at least one α-amino acid residue in the polypeptide is replaced with a β-amino acid residue.

In various embodiments, from 1 to 10 α-amino acid residues in the polypeptide may be replaced with a β-amino acid residue, for example, 1, 2, 3, 4, 5, 6, 7, 8, 9, or 10 α-amino acid residues in the polypeptide are replaced with a β-amino acid residue.

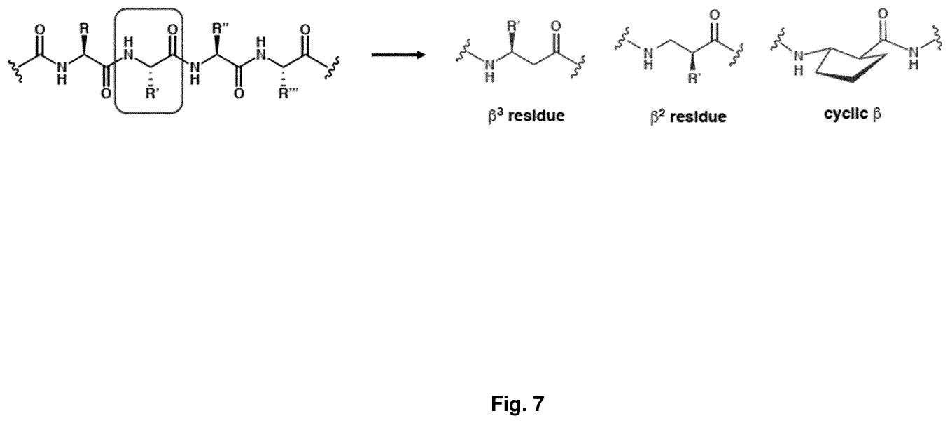

β-amino acid residues may be linear, unsubstituted, or substituted at the α- or 1-position carbon atoms of the backbone (i.e., at the β² or β³ carbon atoms) or may be conformationally constrained by a cyclic group encompassing the a and B backbone carbon atoms of the inserted β-amino acid residue (FIG. 7). Examples of cyclically constrained 1-amino acid residues include 2-aminocyclopentane carboxylic acid (ACPC) and 3-aminopyrrolidine-4-carboxylic acid (APC):

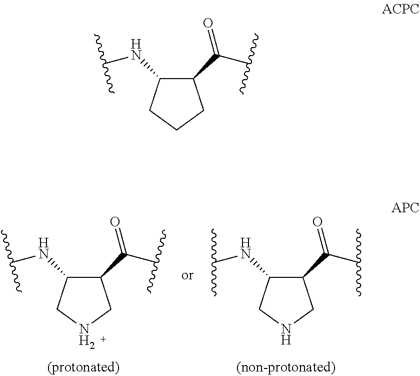 [SMILES: *N[C@H]1CCC[C@@H]1C(*)=O ; *N[C@H]1CNC[C@@H]1C(*)=O ; *N[C@H]1CNC[C@@H]1C(*)=O]

In some embodiments, at least one α-amino acid residue in the polypeptide is replaced with a 2-aminoisobutyric acid (i.e., 2-methylalanine; also known as “Aib”):

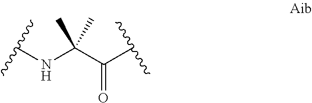

Preferably, the polypeptide disclosed herein further comprises a lipid moiety. Earlier research on lipid-conjugated inhibitory peptides demonstrated that the lipid directs the peptide to cell membranes and increases antiviral efficacy (U.S. Pat. No. 8,629,101 β²; Ingallinella et al. PNAS 2009, 106: 5801; Park and Gallagher, Virology 2017, 511: 9-18). Examples of the lipid moieties include cholesterol, tocopherol, and palmitate. For lipid conjugation, the polypeptide is typically extended at the C terminus, e.g., by a Gly-Ser-Gly-Ser-Gly-Cys segment (SEQ ID NO: 35). The Cysteine side chain is used as a nucleophilic handle to append a lipid moiety, e.g., cholesterol, with an intervening tetra-ethylene glycol segment. The lipid moiety is intended to anchor the peptide in cellular membranes. In some embodiments, at least one α-amino acid residue of the C-terminus segment (e.g., SEQ ID NO: 35) is replaced with a β-amino acid residue.

A poly(ethylene glycol) moiety (e.g., PEG4) can be added between the polypeptide and the lipid moiety. It has been shown that the PEG moiety inserted between the polypeptide and the lipid moiety leads to enhanced broad-spectrum activity and potency (WO2021/216891 A2). In some embodiments, at least one PEG moiety is added between the polypeptide and the lipid moiety.

As shown in Table 1 below, SEQ ID NOs: 5-34 are a series of exemplary polypeptides according to the present disclosure. The polypeptides are derived from SEQ ID NO: 2 and have at least one α-amino acid residue replaced with a 13-amino acid residue. These compounds are exemplary, not exhaustive.

TABLE 1
Sequence List. SEQ ID NOs: 5-34 are exemplary anti-CoV2 polypeptides disclosed
and claimed herein. Bold residues are β²- or β³-amino acid residues that share the
same sidechain as their a-amino acid analogs. The bold, underlined “A” residue is 2-
aminoisobutyric acid (i.e., 2-methylalanine; also known as “Aib”). The “Z” residue is
3-aminopyrrolidine-4-carboxylic acid (also known as “APC”), which may or may not be
protonated.

| Name | Sequence | SEQ ID NO. |
|---|---|---|
| Native CoV2 HRC | DISGINASVVNIQKEIDRLNEVAKNLNESLIDLQEL | 1 |
| Peptide 1 | DISQINASVVNIEYEIKKLEEVAKKLEESLIDLQEL | 2 |
| Peptide 2 | SIDQINATFVDIEYEIKKLEEVAKKLEESYIDLKEL | 3 |
| EK1 | SLDQINVTFLDLEYEMKKLEEAIKKLEESYIDLKEL | 4 |
| D1 D32 | DISQINASVVNIEYEIKKLEEVAKKLEESLIDLQELGSGSGC | 5 |
| S8 D32 | DISQINASVVNIEYEIKKLEEVAKKLEESLIDLQELGSGSGC | 6 |
| D1 Q4 D32 Q34 | DISQINASVVNIEYEIKKLEEVAKKLEESLIDLQELGSGSGC | 7 |
| D1 Q4 D32 E35 | DISQINASVVNIEYEIKKLEEVAKKLEESLIDLQELGSGSGC | 8 |
| D1 S8 D32 E35 | DISQINASVVNIEYEIKKLEEVAKKLEESLIDLQELGSGSGC | 9 |
| Bf APC/B3E | DISQINASVVNIEYEIZKLEEVAZKLEESLIDLQELGSGSGC | 10 |
| Cf APC/B3E | DISQINASVVNIEYEIKZLEEVAKZLEESLIDLQELGSGSGC | 11 |
| Cf B3K/B3E | DISQINASVVNIEYEIKKLEEVAKKLEESLIDLQELGSGSGC | 12 |
| Cf APC/aAPC | DISQINASVVNIEYEIKZLEUVAKZLEUSLIDLQELGSGSGC | 13 |
| E21 Z24 E28 | DISQINASVVNIEYEIKKLEEVAZKLEESLIDLQELGSGSGC | 14 |
| Z17 Z24 E28 | DISQINASVVNIEYEIZKLEEVAZKLEESLIDLQELGSGSGC | 15 |
| Z17 E21 E28 | DISQINASVVNIEYEIZKLEEVAKKLEESLIDLQELGSGSGC | 16 |
| Z17 E21 Z24 | DISQINASVVNIEYEIZKLEEVAZKLEESLIDLQELGSGSGC | 17 |
| α/β-SARS2 HRC QE-1 | DISQINASVVNIEYEIZKLEEVAZKLEESLIDLQELGSGSGC | 18 |
| α/β-SARS2 HRC QE-2 | DISQINASVVNIEYEIZKLEEVAKZLEESLIDLQELGSGSGC | 19 |
| α/β-SARS2 HRC QE-3 | DISQINASVVNIEYEIZKLEEVAZKLEESLIDLQELGSGSGC | 20 |
| α/β-SARS2 HRC QE-4 | DISQINASVVNIEYEIZKLEEVAZKLEESLIDLQELGSGSGC | 21 |
| α/β-SARS2 HRC QE-5 | DISQINASVVNIEYEIZKLEEVAKKLEESLIDLQELGSGSGC | 22 |
| α/β-SARS2 HRC QE-6 | DISQINASVVNIEYEIKKLEEVAZKLEESLIDLQELGSGSGC | 23 |
| α/β-SARS2 HRC QE-7 | DISQINASVVNIEYEIZKLEEVAZKLEESLIDLQELGSGSGC | 24 |
| α/β-SARS2 HRC QE-8 | DISQINASVVNIEYEIZKLEEVAZKLEESLIDLQELGSGSGC | 25 |
| α/β-SARS2 HRC QE-9 | DISQINASVVNIEYEIZKLEEVAZKLEESLIDLQELGSGSGC | 26 |
| α/β-SARS2 HRC QE-10 | DISQINASVVNIEYEIZKLEEVAZKLEESLIDLQELGSGSGC | 27 |
| α/β-SARS2 HRC QE-11 | DISQINASAVNIEYEIZKLEEVAZKLEESLIDLQELGSGSGC | 28 |
| α/β-SARS2 HRC QE-12 | DISQINASVVNIEYEIZKLEEVAZKLEESLIDLQELGSGSGC | 29 |
| α/β-SARS2 HRC QE-13 | DISQINASVVNIEYEIZKLEEVAZKLEESLIDLQELGSGSGC | 30 |
| α/β-SARS2 HRC QE-14 | DISQINASVVNIEYEIZKLEEVAZKLEESLIDLQELGSGSGC | 31 |
| α/β-SARS2 HRC QE-15 | DISQINASVVNIEYEIZKLEEVAZKLEESLIDLQELGSGSGC | 32 |
| α/β-SARS2 HRC QE-16 | DISQINASVVNIAYEIZKLEEVAZKLEESLIDLQELGSGSGC | 33 |
| α/β-SARS2 HRC QE-17 | DISQINASVVNIEYEIZKLEEVAZKLEESLIDLQELGSGSGC | 34 |
| C-terminus linker | GSGSGC | 35 |

### Pharmaceutical Compositions

Also disclosed herein are pharmaceutical compositions comprising the anti-CoV2 polypeptides or a pharmaceutically suitable salt thereof as described herein. More specifically, the pharmaceutical composition may comprise one or more of the anti-CoV2 polypeptides as well as a standard, well-known, non-toxic pharmaceutically suitable carrier, adjuvant or vehicle such as, for example, phosphate buffered saline, water, ethanol, polyols, vegetable oils, a welling agent or an emulsion such as a water/oil emulsion. The composition may be in either a liquid, solid or semi-solid form. For example, the composition may be in the form of a tablet, capsule, ingestible liquid or powder, injectable, suppository, or topical ointment or cream. Proper fluidity can be maintained, for example, by maintaining appropriate particle size in the case of dispersions and by the use of surfactants. It may also be desirable to include isotonic agents, for example, sugars, sodium chloride, and the like. Besides such inert diluents, the composition may also include adjuvants, such as wetting agents, emulsifying and suspending agents, sweetening agents, flavoring agents, perfuming agents, and the like.

Suspensions, in addition to the active compounds, may comprise suspending agents such as, for example, ethoxylated isostearyl alcohols, polyoxyethylene sorbitol and sorbitan esters, microcrystalline cellulose, aluminum metahydroxide, bentonite, agar-agar and tragacanth or mixtures of these substances.

Solid dosage forms such as tablets and capsules can be prepared using techniques well known in the art of pharmacy. For example, the anti-CoV2 polypeptides produced as described herein can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant polypeptides.

For intravenous administration, the polypeptides may be incorporated into commercial formulations. Where desired, the individual components of the formulations may be provided individually, in kit form, for single or multiple use.

The pharmaceutical compositions may be administered orally. For example, a liquid preparation may be administered orally. Additionally, a homogenous mixture can be completely dispersed in water, admixed under sterile conditions with physiologically acceptable diluents, preservatives, buffers or propellants in order to form a spray or inhalant. The route of administration will, of course, depend upon the desired effect and the medical stated of the subject being treated. The dosage of the composition to be administered to the patient may be determined by one of ordinary skill in the art and depends upon various factors such as weight of the patient, age of the patient, immune status of the patient, etc., and is ultimately at the discretion of the medical professional administering the treatment.

With respect to form, the composition may be, for example, a solution, a dispersion, a suspension, an emulsion or a sterile powder which is then reconstituted. The composition may be administered in a single daily dose or multiple doses.

The present disclosure also includes treating CoV2 in mammals, including humans, by administering an inhibiting and/or CoV2 symptom-ameliorating amount of one or more of the anti-CoV2 polypeptides described herein. In particular, the compositions of the present disclosure may be used to treat CoV2 conditions of any and all description.

It should be noted that the above-described pharmaceutical compositions may be utilized in connection with non-human animals, both domestic and non-domestic, as well as humans.

### Examples

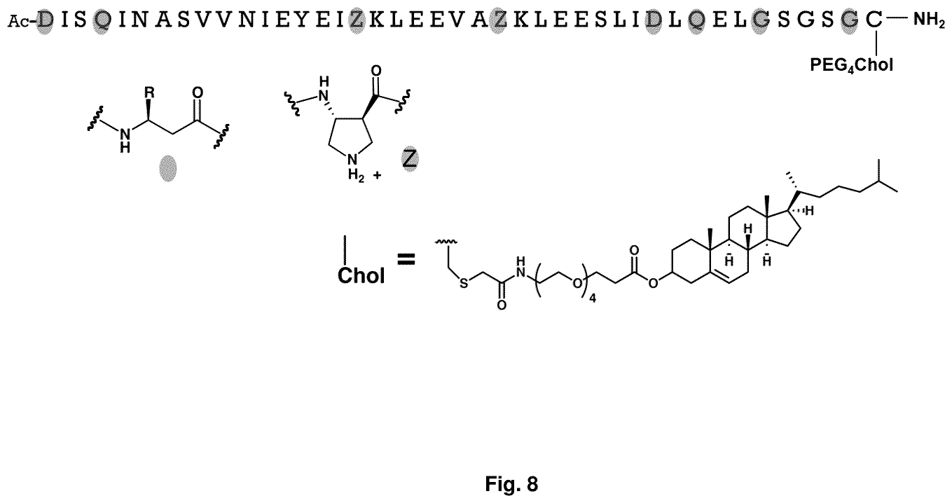

In this Example, exemplary anti-CoV2 polypeptides were evaluated in inhibiting CoV2 S-protein-mediated fusion. The polypeptides tested herein include aliphatic β-amino acid substitutions and/or cyclically constrained β-amino acid substitutions, and are linked to a poly(ethylene glycol)-cholesterol “tail” through an C-terminal linker of “GSGSGC” (SEQ ID NO: 35). FIG. 8 shows one of the exemplary polypeptides having a poly(ethylene glycol)-cholesterol “tail.”

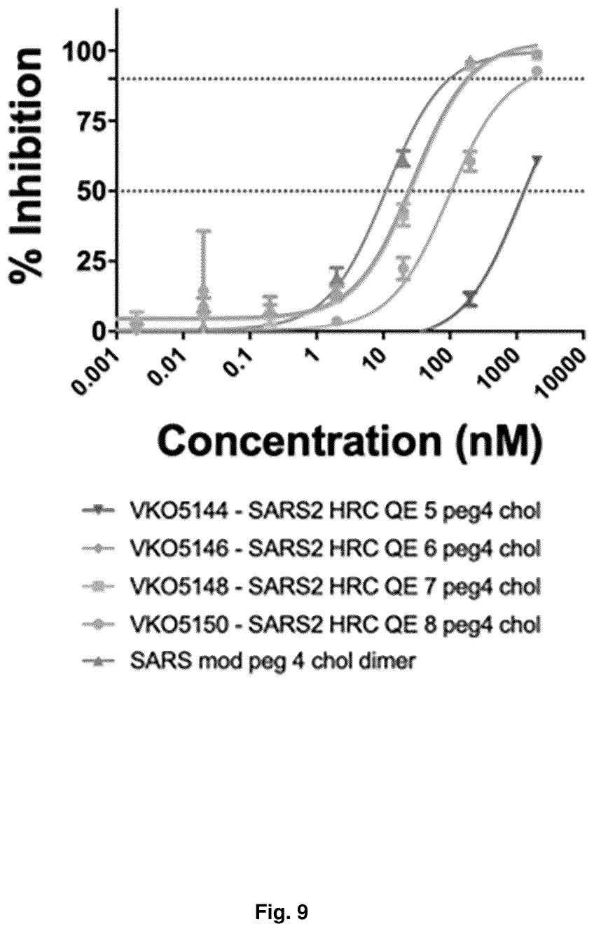

FIG. 9 shows results from cell-cell fusion assays, where percent inhibition corresponds to the extent of suppression of the luminescence signal that is observed in the absence of any inhibitor (i.e., 0% inhibition corresponds to maximum luminescence signal). The SARS2 HRC QE6 and QE7 (SEQ ID NOs: 23-24) potently inhibited S-mediated fusion, with 50% inhibitory concentration (IC₅₀) of about 20 nM and 90% inhibitory concentration (IC₉₀) of about 100 nM. SARS2 HRC QE 8 (SEQ ID NO: 25) also shows efficacy in inhibiting S-mediated fusion, with IC₅₀ of about 100 nM. SARS2 HRC QE 5 (SEQ ID NO: 22) is less effective, with IC₅₀ of about 1000 nM.

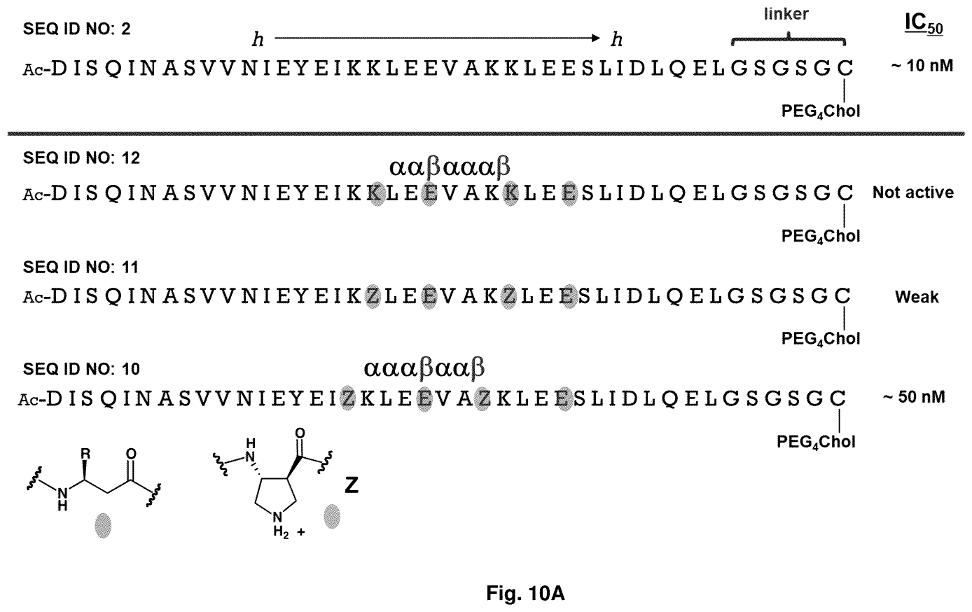

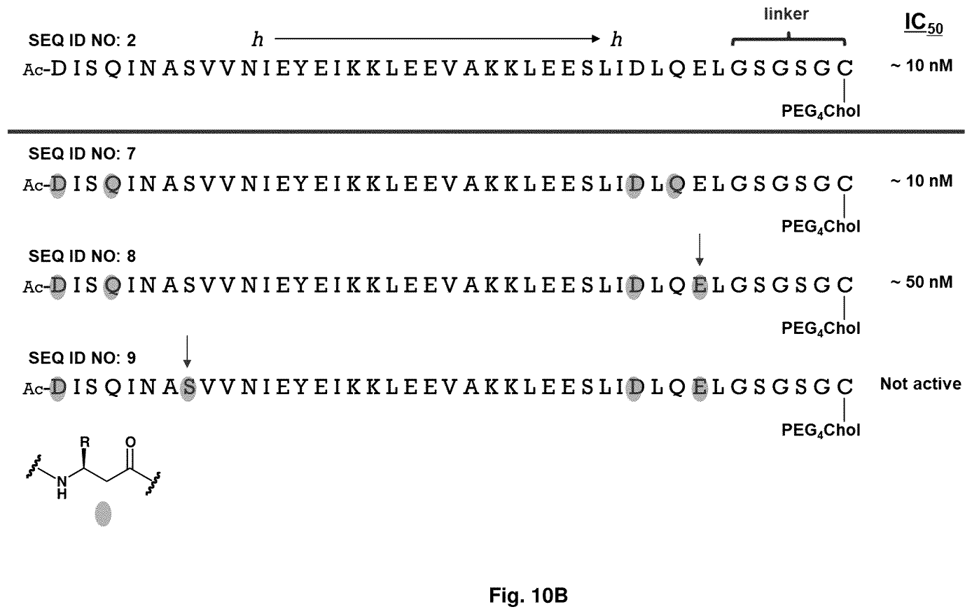

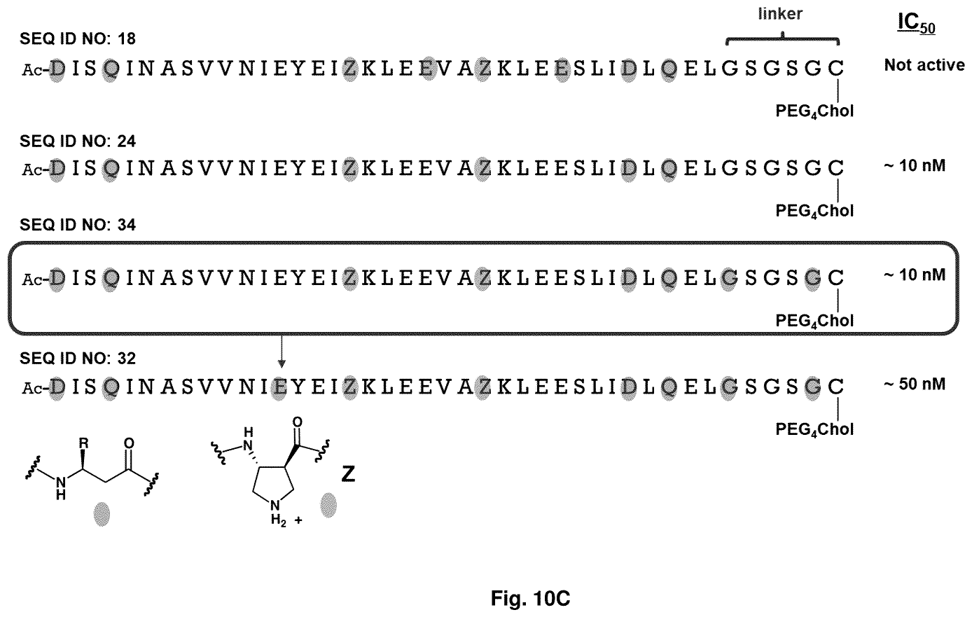

FIGS. 10A-10C shows more results from cell-cell fusion assays, testing a wider range of exemplary anti-CoV2 polypeptides. Polypeptides of SEQ ID NOs: 7, 24, and 34 show the greatest potency in inhibiting S-mediated fusion, with IC₅₀ of about 10 nM, followed by the polypeptides of SEQ ID NOs: 8, 10, and 32, with IC₅₀ of about 50 nM.

### Methods

Peptide Synthesis. Peptides were prepared on NovaPEG rink amide resin (NovaBiochem, a wholly owns subsidiary of Merck KGaA, Darmstadt, Germany) using previously reported microwave-assisted conditions for Fmoc-based solid-phase peptide synthesis. See Home, W. S., Boersma, M. D., Windsor, M. A. & Gellman, S. H. Sequence-Based Design of a/β-Peptide Foldamers that Mimic α-Helical BH3 Domains, Angew. Chem. Int. Ed. 47, 2853-6, (2008); Home, W. S., Johnson, L. M., Ketas, T. J., Klasse, P. J., Lu, M., Moore, J. P., Gellman, S. H. Structural and biological mimicry of protein surface recognition by a/p-peptide foldamers. Proc. Natl Acad. Sci. USA 106, 14751-6, (2009); Johnson, L. M., Mortenson, D. E., Yun, H. G., Home, W. S., Ketas, T. J., Lu, M., Moore, J. P., & Gellman, S. H. Enhancement of α-Helix Mimicry by an α/β-Peptide Foldamer via Incorporation of a Dense Ionic Side-Chain Array. J Am. Chem. Soc. 134, 7317-20, (2012); Boersma, M. D., Haase, H. S., Peterson-Kaufman, K. J., Lee, E. F., Clarke, O. B., Colman, P. M., Smith, B. J., Home, W. S., Fairlie, W. D., & Gellman, S. H. Evaluation of diverse α/β-backbone patterns for functional α-helix mimicry: analogues of the Bim BH3 domain. J. Am. Chem. Soc. 134, 315-23, (2012); and Home, W. S., Price, J. L., & Gellman, S. H. Interplay among side chain sequence, backbone composition, and residue rigidification in polypeptide folding and assembly. Proc. Natl Acad Sci USA 105, 9151-6, (2008).

After the chain had been assembled, peptides were cleaved from the resin and side chains were deprotected by treating the resin with 2 mL trifluoroacetic acid (TFA), 50 μL water, and 50 μL triisopropylsilane for 3 hrs. The TFA solution is then dripped into cold ether to precipitate the deprotected peptide. Peptides were purified on a prep-C18 column (Sigma-Aldrich, St. Louis, MO) using reverse phase-HPLC. Purity was assessed by RP-HPLC (solvent A: 0.1% TFA in water, solvent B: 0.1% TFA in acetonitrile, C18 analytical column (4.6×250 mm), flow rate 1 mL/min, gradient 10-60% B solvent over 50 minutes). Masses were measured by MALDI-TOF-MS. (Data not shown.)

Protease Assays. An HPLC method from the literature was used to assess protease action on selected compounds. See Murage, E. N., Gao, G. Z., Bisello, A., & Ahn, J. M. Development of Potent Glucagon-like Peptide-1 Agonists with High Enzyme Stability via Introduction of Multiple Lactam Bridges. J. Med. Chem. 53, 6412-20, (2010).

Two nmol of solid peptide were dissolved in 40 μL of TBS pH 8.0 (resulting concentration of peptide=40 M) before protease was added. Chymotrypsin was purchased from Promega (Fitchburg, WI; catalog #V1062), and neprilysin was purchased from Reprokine. Ltd. (Valley Cottage, NY; catalog #RKP08473); stock solutions of 250 μg/mL chymotrypsin and 200 μg/mL neprilysin in water were prepared. A 10 μL aliquot of protease stock solution was added to 40 μL of 40 μM peptide solution to begin the reaction. Periodically, a 10 μL aliquot of the solution was removed, and protease action was halted by adding this aliquot to 100 μL of 1% aqueous TFA solution. A portion (100 μL) of the quenched solution was injected onto an HPLC column using the conditions described under “Peptide Synthesis”, and peaks were analyzed using MALDI-TOF MS. The time course of peptide degradation was experimentally determined by integrating the area of each peak in a series of HPLC traces. The area percent of parent peptide (relative to the initial trace) was calculated for each trace and plotted in GraphPad Prism as an exponential decay to determine half-life values.

Cells. Human embryonic kidney (HEK) 293T and Vero (African green monkey kidney) cells were grown in Dulbecco's modified Eagle's medium (DMEM; Invitrogen; Thermo Fisher Scientific) supplemented with 10% fetal bovine serum (FBS) and antibiotics in 5% CO₂. Vero E6 cells (ATCC CRL-1586) were grown in minimum essential medium with Earle's salts (EMEM; Gibco) supplemented with 6% FBS and antibiotics in 5% CO₂.

Plasmids. The cDNAs coding for hACE2 fused to the fluorescent protein Venus, dipeptidyl peptidase 4 (DPP4) fused to the fluorescent protein Venus and SARS-CoV-2 S (codon optimized for mammalian expression) were cloned in a modified version of the pCAGGS (with puromycin resistance for selection).

Viruses. SARS-CoV-2 strain USA_WA1/2020 was obtained from the University of Texas Medical Branch (UTMB) World Reference Center for Emerging Viruses and Arboviruses (WRCEVA) and propagated in Vero E6 cells. Virus stocks were generated from clarified cell culture supernatants harvested 3 or 4 days postinoculation. The recombinant virus expressing neon green (icSARS-CoV-2-mNG) was developed by Pei-Yong Shi and colleagues (Xie X. et al., An infectious cDNA clone of SARS-CoV-2. Cell Host Microbe 27: 841-848.e3, 2020) and propagated in Vero E6 cells. All work with infectious virus (propagation, titration, and plaque reduction assays) was done in the biosafety level 3 (BSL3) facility at the Galveston National Laboratory of UTMB.

β-Gal complementation-based fusion assay (Cell-cell fusion assay). We previously adapted a fusion assay based on alpha complementation of -galactosidase (β-Gal) (Porotto M. et al., Inhibition of Nipah virus infection in vivo: targeting an early stage of paramyxovirus fusion activation during viral entry. PLoS Pathog 6: e1001168, 2010). In this assay, hACE2 or DDP4 receptor-bearing cells expressing the omega peptide of β-Gal are mixed with cells coexpressing glycoprotein S and the alpha peptide of β-Gal, and cell fusion leads to alpha-omega complementation. Fusion is stopped by lysing the cells, and after addition of the substrate (Tropix Galacto-Star chemiluminescent reporter assay system; Applied Biosystem), luminescence is quantified on a Tecan M1000PRO microplate reader.

Viral titration and plaque reduction neutralization assay. Titers of virus stocks were determined by plaque assay in Vero E6 cells grown in six-well tissue culture plates. Virus stocks were serially diluted 10-fold in PBS, and 0.2 ml of each dilution was inoculated into quadruplicate wells and allowed to adsorb at 37° C. for 1 h with rocking every 15 min. Monolayers were rinsed with Dulbecco's phosphate-buffered saline (DPBS; Coming) and then overlaid with a semisolid medium containing MEM, 5% FBS, antibiotics, and ME agarose (0.6%). Cultures were incubated at 37° C. for 3 days and overlaid with DPBS containing neutral red (3.33 g/liter; Thermo Fisher Scientific) as a stain (10%), and plaques were counted after 4 to 5 h.

Peptides were tested for inhibitory activity against SARS-CoV-2 by plaque reduction neutralization assay. Peptides were serially diluted in molecular biology grade water (10,000 nM through 5 nM or 1,000 nM through 0.5 nM), each peptide dose was mixed with an equal volume of virus containing 500 particle-forming units (PFU)/ml in MEM, and the peptide/virus mixtures were incubated at 37° C. for 1 h. Each peptide dose/virus mixture was inoculated into triplicate wells of Vero E6 cells in six-well plates (0.2 ml per well) and allowed to adsorb at 37° C. for 1 h with rocking every 15 min. Monolayers were rinsed with DPBS prior to the addition of medium overlay containing MEM, 5% FBS, antibiotics, and ME agarose (0.6%). Cultures were incubated at 37° C. for 3 days and overlaid with medium containing neutral red as a stain, and plaques were counted after 4 to 5 h. Virus controls were mixed with sterile water instead of peptide

HAE cultures. The EpiAirway AIR-100 system (MatTek Corporation) consists of normal human-derived tracheo/bronchial epithelial cells that have been cultured to form a pseudostratified, highly differentiated mucociliary epithelium closely resembling that of epithelial tissue in vivo. Upon receipt from the manufacturer, HAE cultures were handled as we have done previously (Outlaw V. K. et al., Dual inhibition of human parainfluenza type 3 and respiratory syncytial virus infectivity with a single agent. J Am Chem Soc 141: 12648-12656. 2019; Moscona A. et al., A recombinant sialidase fusion protein effectively inhibits human parainfluenza viral infection in vitro and in vivo. J Infect Dis 202: 234-241, 2010; Palermo L. M. et al., Human parainfluenza virus infection of the airway epithelium: the viral hemagglutinin-neuraminidase regulates fusion protein activation and modulates infectivity. J Virol 83: 6900-6908, 2009). Briefly, cultures were transferred to six-well plates containing 1.0 ml medium per well (basolateral feeding, with the apical surface remaining exposed to air) and acclimated at 37° C. in 5% CO₂ for 24 h prior to experimentation.

Viral infection of HAE. HAE cultures were infected by applying 200 μl of EpiAirway phosphate-buffered saline (MatTek TEER buffer) containing 2,000 PFU of infectious-clone-derived SARS-CoV-2 expressing a stable mNeonGreen reporter gene (icSARS-CoV-2-mNG) (Xie X. et al., An infectious cDNA clone of SARS-CoV-2. Cell Host Microbe 27: 841-848.e3, 2020) to the apical surface for 90 min at 37° C. At 90 min, the medium containing the inoculum was removed, the apical surface was washed with 200 μl of TEER buffer, and either 20 μl of peptide (10,000 nM) or an equivalent amount of TEER buffer was added as a treatment. Cultures were fed each day by replenishing 1.0 ml medium on the basolateral side after harvest. The final peptide concentration was 200 nM.

Virus was harvested by adding 200 μl TEER buffer per well to the HAE cultures' apical surface and allowed to equilibrate for 30 min at 37° C. The suspension was then collected, inactivated with TRIzol reagent (Thermo Fisher) and processed for RT-qPCR. This viral collection was performed sequentially with the same wells of cells on each day postinfection. After harvest of apical and basolateral suspensions, cells were lysed using TRIzol on day 7 postinfection. The amount of infectious virus from HAE supernatants collected from apical and basolateral sides were determined by plaque assay in Vero E6 cells grown in 12-well plates inoculated with 0.1 ml per well (triplicates of each 10-fold dilution in PBS).

Quantitative RT-PCR. Viral titers in cell extracts and supernatant fluid were estimated by quantitative RT-PCR (RT-qPCR). Total RNA was extracted using RNeasy minikit according to the manufacturer's instructions (Qiagen). Reverse transcriptions were performed using GoScript reverse transcription system (Promega). Obtained cDNAs were diluted 1:10. Quantitative PCR (qPCR) was performed using Platinum SYBR Green qPCR SuperMix-UDG with ROX kit (Invitrogen). qPCR was run on the ABI 7000 PCR system (Applied Biosystems) using the following protocol: (i) 5 min at 95° C.; (ii) 40 cycles with 1 cycle consisting of 15 s at 95° C. and 1 min at 60° C.; (iii) f a melting curve up to 95° C. at 0.8° C. intervals. A standard reference (2019-nCoV Positive Control-nCoVPC from “the CDC 2019-nCoV Real-Time” kit) was included in each run to standardize results.

Cell toxicity assay. HEK293T or Vero cells were incubated with the indicated concentration of the peptides or vehicle (dimethyl sulfoxide) at 37° C. The cytotoxicity was determined after 24 h using the Vybrant MTT cell proliferation assay kit according to the manufacturer's guidelines. The absorbance was read at 540 nm using Tecan M1000PRO microplate reader. HAE cultures were incubated at 37° C. in the presence or absence of 1, 10, or 100 M concentrations of the peptide. The peptide was added to the feeding medium. Cell viability was determined on day 7 using the Vybrant MTT cell proliferation assay kit according to the manufacturer's guidelines. The absorbance was read at 540 using a Tecan M1000PRO microplate reader.

What is claimed is:

1. A composition of matter comprising a polypeptide as shown in SEQ ID NO: 2, or a polypeptide with at least 80%, 85%, 90%, or 95%, but less than 100% sequence identity to SEQ ID NO: 2, wherein at least one α-amino acid residue in the polypeptide is replaced with a β-amino acid residue.

2. The composition of matter of claim 1, wherein from 1 to 10 α-amino acid residues in the polypeptide are replaced with a β-amino acid residue.

3. The composition of matter of claim 1, wherein at least one α-amino acid residue in the polypeptide is replaced with a cyclically constrained β-amino acid residue.

4. The composition of matter of claim 1, wherein at least one α-amino acid residue in the polypeptide is replaced with a cyclically constrained β-amino acid residue selected from the group consisting of 2-aminocyclopentane carboxylic acid and β-aminopyrrolidine-4-carboxylic acid.

5. The composition of matter of claim 1, wherein at least one α-amino acid residue in the polypeptide is replaced with a 2-aminoisobutyric acid.

6. The composition of matter of claim 1, wherein the polypeptide further comprises a lipid moiety.

7. The composition of matter of claim 6, wherein the lipid moiety is attached to a terminus of the polypeptide.

8. The composition of matter of claim 6, wherein the lipid moiety is selected from the group consisting of cholesterol, tocopherol, and palmitate.

9. The composition of matter of claim 1, wherein the polypeptide further comprises at least one poly(ethylene glycol) moiety.

10. The composition of matter of claim 1, wherein the polypeptide further comprises a lipid moiety and at least one poly(ethylene glycol) moiety.

11. The composition of matter of claim 10, wherein the poly(ethylene glycol) moiety is between the polypeptide and the lipid moiety.

12. The composition of matter of claim 10, wherein the lipid moiety is selected from the group consisting of cholesterol, tocopherol, and palmitate.

13. The composition of matter of claim 1, wherein the polypeptide comprises a compound selected from the group consisting of SEQ. ID. NOS:5-34.

14. A composition of matter comprising SEQ ID NO: 34, or a polypeptide with at least 80%, 85%, 90%, or 95% sequence identity thereto, but less than 100% sequence identity to SEQ ID NOs: 7, 23, 24, 32, and 34.

15. A method to inhibit infection by CoV2 in a mammalian subject, including a human subject, the method comprising administering to the subject a CoV2 infection-inhibiting amount of a composition of matter as recited in claim 1.

16. A method to ameliorate symptoms of CoV2 infection in a mammalian subject, including a human subject, the method comprising administering to the subject a CoV2 symptom-ameliorating amount of a composition of matter as recited in claim 1.

17. A pharmaceutical composition comprising a composition of matter as recited in claim 1, in combination with a pharmaceutically suitable carrier.
